(19) United States
(12) Patent Application Publication
(10) Pub. No.: US 2015/0322499 A1
(43) Pub. Date: Nov. 12, 2015

(54) METHOD FOR ANALYZING MOLECULE USING THERMOPHORESIS

(71) Applicant: NATIONAL YANG-MING UNIVERSITY, Taipei City (TW)

(72) Inventors: Yih-Fan Chen, Taipei (TW); Li-Hsien Yu, Tainan City (TW)

(21) Appl. No.: 14/539,439

(22) Filed: Nov. 12, 2014

(30) Foreign Application Priority Data
May 12, 2014 (TW) 103116655

(51) Int. Cl. C12Q 1/68; G01N 33/68; G01N 33/558; G01N 33/53

(52) U.S. Cl. CPC C12Q 1/6825; G01N 33/5308; G01N 33/6866; G01N 33/558; C12Q 1/6832

(57) ABSTRACT

A method for analyzing a target molecule using thermophoresis is provided. The method of the invention comprises (1) providing a solution containing samples, labeled molecules, and probe particles; (2) providing a temperature control system to create a temperature gradient within the solution; (3) detecting the expression level of the labeled molecule in a predetermined area and a contrast area; and (4) analyzing the difference in the expression level of the labeled molecules between the predetermined area and the contrast area to determine the result. In another embodiment of the invention, the solution contains samples and “labeled molecule-reactant-probe particle” complexes. In the present invention, the probe particles are used to increase the difference in thermophoresis between the molecular complexes and the free labeled molecules, which can improve the accuracy of the quantification of the target molecules using thermophoresis.

### CROSS-REFERENCE TO RELATED APPLICATIONS

This Non-provisional application claims priority under 35 U.S.C. §119(a) on Patent Application No(s). 103116655 filed in Taiwan, Republic of China May 12, 2014, the entire contents of which are hereby incorporated by reference.

### FIELD OF THE INVENTION

The present invention relates to a method for analysis of molecule using thermophoresis, and in particular relates to a method for determining an amount of nucleic acids and proteins using thermophoresis.

### DESCRIPTION OF THE RELATED ART

Thermophoresis is the directed movement of particles in a temperature gradient. Microscale thermophoresis (MST) is a method for analyzing biomolecules using thermophoresis. Changes in the properties of molecules (e.g., size, charge, hydration shell and solvation entropy of molecules) due to the binding between molecules change molecules' thermophoresis. MST can measure the binding affinity between molecules based on molecules' thermophoretic motion. MST allows measurement of interactions directly in solution without immobilizing molecules to a surface.

Duhr el al. in Eur. Phys. J. E 15; 277-286, 2004 “Thermophoresis of DNA determined by microfluidic fluorescence” discusses an optical approach to measure thermophoresis of biomolecules in small flow chambers.

Molecules can move along the temperature gradient because of thermophoresis. Binding between molecules can affect molecules' thermophoretic motion, but the change in the motion is usually not very significant. Therefore, when thermophoretic motion of molecules is measured by observing the spatial distribution of fluorescence in the temperature gradient, the change in the distribution of fluorescence with the fraction of bound complexes is usually small. If the binding between molecules only causes a small change in thermophoresis, it is difficult to measure the concentration of molecules based on thermophoretic motion of molecules.

### BRIEF SUMMARY OF INVENTION

To overcome the problem mentioned above, the present invention provides a probe particle to produce a significant difference in thermophoretic mobility between molecular complexes and free fluorescent molecules. The probe particle of the present invention can improve the accuracy of molecule detection. The probe particle of the present invention can be used to analyze DNA, RNA, proteins, or organic or inorganic molecules.

The invention provides a method for analyzing a target molecule in a sample using thermophoresis. The method comprises (1) providing a solution comprising samples, labeled molecules, and probe particles in an accommodating space; (2) providing a temperature control system in a control region of the accommodating space to create a temperature gradient within the solution; (3) detecting the expression level of the labeled molecules in a predetermined area and a contrast area; and (4) analyzing the difference in the expression level of the labeled molecules between the predetermined area and the contrast area. The probe particles can bind to the target molecules. If the sample contains target molecules, then the probe particles, the target molecules, and the labeled molecules can form “probe particle-target molecule-labeled molecule” complexes. The direction or speed of the motion of the molecular complexes in a temperature gradient is different from that of the free labeled molecules.

In one embodiment, the probe particle is a nanoparticle with at least one probe attached on a surface thereof.

In one embodiment, the probe is a DNA molecule, a RNA molecule, or an antibody.

In one embodiment, the nanoparticle is sensitive to thermophoresis.

In one embodiment, the nanoparticle is a metal, plastic, glass, oxide or semiconductor nanoparticle.

In one embodiment, the nanoparticle is a gold nanoparticle.

In one embodiment, the target molecule is a DNA molecule, a RNA molecule, or a protein.

In one embodiment, the labeled molecule is a DNA molecule, a RNA molecule, or an antibody labeled with fluorophores or dyes.

In one embodiment, the expression level of the labeled molecules is the intensity of fluorescence.

In one embodiment, the heating or cooling is conducted by a temperature control system.

In one embodiment, the heating is conducted by an electrode or a light emitting device of a temperature control system.

In one embodiment, the accommodating space is a microchamber or a capillary tube.

The invention provides a method for analyzing a target molecule in a sample using thermophoresis. The method comprises (1) providing a solution comprising samples and “labeled molecule-reactant-probe particle” complexes in an accommodating space; (2) providing a temperature control system in a control region of the accommodating space to create a temperature gradient within the solution; (3) detecting the expression level of the labeled molecules in a predetermined area and a contrast area; and (4) analyzing the difference in the expression level of the labeled molecules between the predetermined area and the contrast area to determine the result. The direction or speed of the motion of the molecular complexes in the temperature gradient is different from that of the free labeled molecules, and the binding affinity of the reactants to the target molecules is higher than the labeled molecules and the probe particles. If the sample contains the target molecules, the reactants bind to the target molecules and disrupt the labeled “molecule-reactant-probe particle” complexes.

In one embodiment, the probe particle is a nanoparticle with at least one probe attached on a surface thereof.

In one embodiment, the probe is a DNA molecule, a RNA molecule, or an antibody.

In one embodiment, the nanoparticle is sensitive to thermophoresis.

In one embodiment, the nanoparticle is a metal, plastic, glass, oxide or semiconductor nanoparticle.

In one embodiment, the nanoparticle is a gold nanoparticle.

In one embodiment, the target molecule is a DNA molecule, a RNA molecule, or a protein.

In one embodiment, the labeled molecule is a DNA molecule, a RNA molecule, or an antibody labeled with fluorophores dyes.

In one embodiment, the expression level of the labeled molecules is the intensity of fluorescence.

In one embodiment, the heating or cooling is conducted by a temperature control system.

In one embodiment, the heating is conducted by an electrode or a light emitting device of the temperature control system.

In one embodiment, the accommodating space is a microchamber or a capillary tube.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIG. 1 is a schematic diagram showing the steps involved in the analysis of a target molecule according to one embodiment of the present invention;

FIG. 2 illustrates the changes in the expression level of the labeled molecules with the concentration of the target molecules.

FIGS. 3A-3B illustrate the relative positions of the control region, the predetermined area, and the contrast area of the invention.

FIG. 4 illustrates a schematic diagram (sandwich method) according to one embodiment of the invention.

FIG. 5 illustrates a schematic diagram showing the steps involved in the analysis of a target molecule according to another embodiment of the present invention

FIG. 6 illustrates a schematic diagram (competition method) according to one embodiment of the invention.

FIG. 7 illustrates that the difference in the intensity of fluorescence between the predetermined area and the contrast are increases with the increased concentration of the target DNA molecule, DNA_{—}3.

FIG. 8 illustrates the difference in the intensity of fluorescence between the predetermined area and the contrast decreases with the target protein concentration.

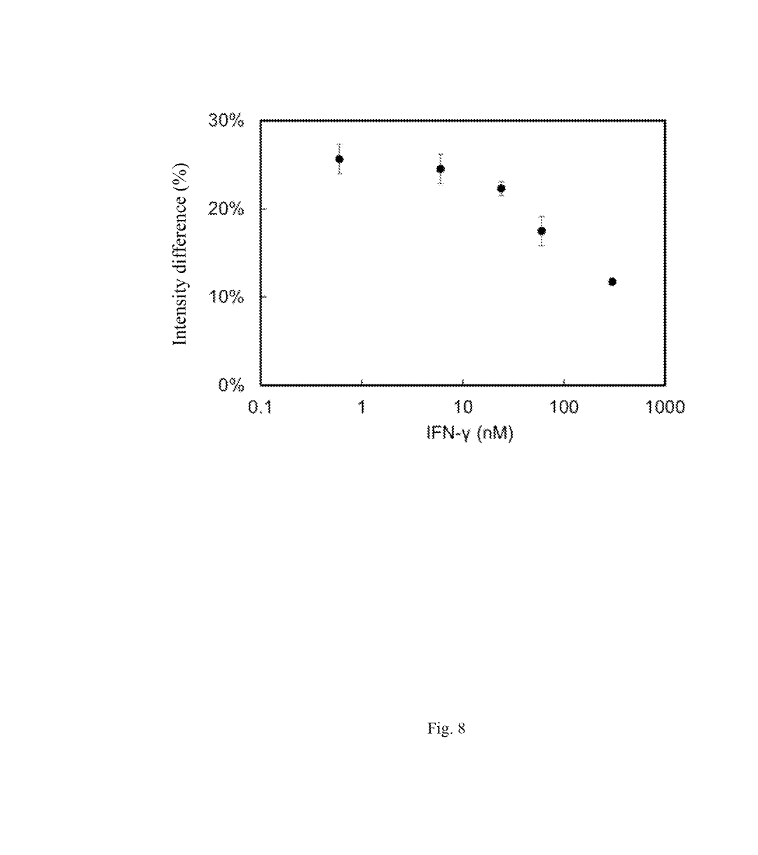

### DETAILED DESCRIPTION OF INVENTION

The following description is of the best-contemplated mode of carrying out the invention. This description is made for illustrating the general principles of the invention and should not be taken in a limited sense. The scope of the invention is best determined by reference to the appended claims.

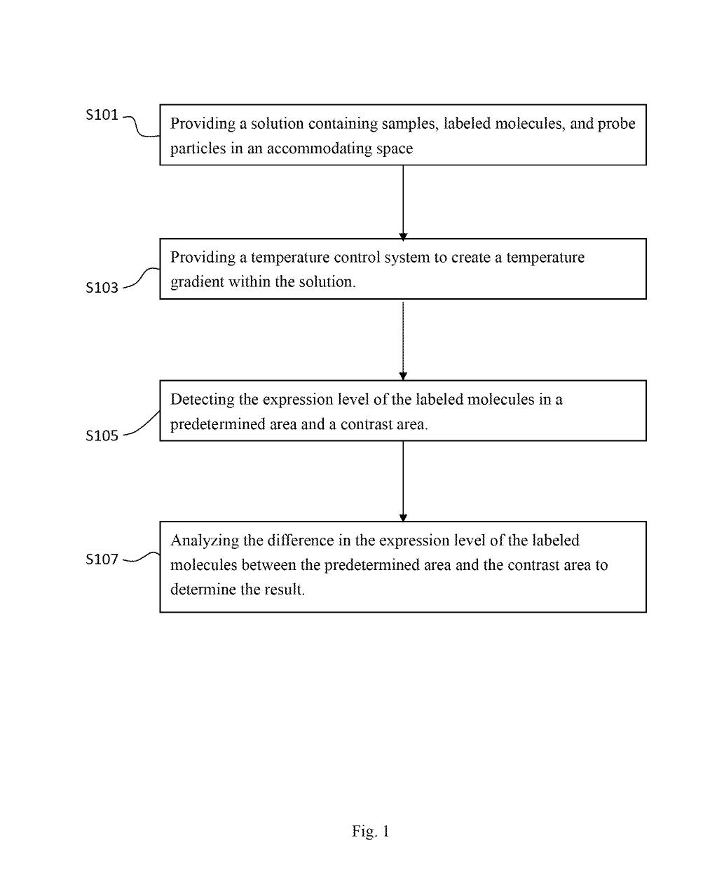

In one aspect of the invention, a method for analyzing a target molecule in a sample using thermophoresis is provided. In the first aspect of the present invention, a method of the invention is shown in FIG. 1. Referring to step S101, a solution is provided in an accommodating space, wherein the solution contains samples, labeled molecules, and probe particles.

The “target molecule” of the invention refers to nucleic acids (e.g., DNA, RNA, LNA, or PNA), proteins, organic or inorganic molecules (e.g., heavy metal ions) or the like.

The “sample” of the invention refers to any sample containing nucleic acids. The biological sample of the invention is not limited and includes any material containing nucleic acids, chromosomes, and/or plasmids. In one embodiment, the biological sample of the invention can be a fungus, a virus, a microorganism, a cell, a blood sample, an amniotic fluid, a cerebrospinal fluid, or a tissue sample from skin, muscle, buccal, conjunctival mucosa, placenta, or gastrointestinal tract. In another embodiment, the sample of the invention can be food, water, or soil.

The “labeled molecule” of the invention refers to a DNA molecule, a RNA molecule, a peptide, an antibody, a protein, or the like labeled with fluorophores or dyes. The fluorophores or dyes include, but are not limited to, fluorescein isothiocyanate (FITC), luciferase, fluorescent protein, chloramphenicol acetyl transferase, or β-galactosidase.

The “probe particle” of the invention refers to a particle linked to a probe. The “particle” or “nanoparticle” of the invention can be a metal or metal oxide, but is not limited thereto. Examples of the particle or nanoparticle include phosphorous, gold, silver, titanium oxide, zinc oxide, or zirconium oxide particle, preferably gold particle. In another embodiment, the particle or nanoparticle can also be non-metal, such as plastic, glass, or polymer. The particle or nanoparticle of the invention can be a commercial product. The size of the “particle” is less than 10,000 nm, preferably, 500 nm, 100 nm, 90 nm, 80 nm, 70 nm, 60 nm, 50 nm, 40 nm, 30 nm, 20 nm or 10 nm.

The “accommodating space” of the invention refers to a space for accommodating a liquid. The space can accommodate at least 0.1 μl of a liquid, preferably, more than 0.5 μl, 1 μl, 1.5 μl, 2 μl, 2.5 μl, 3 μl, or 3.5 μl. For microscopic observation and laser heating, the space preferably is formed by a transparent material such as glass, indium tin oxide (ITO), quartz, metal, or plastic.

If the target molecule is present in the sample, the target molecules, labeled molecules, and probe particles can form “probe particle-target molecule-labeled molecule” complexes. The speed or direction of the motion of the molecular complexes in the temperature gradient is different from that of the free labeled molecules. In one embodiment, the probe particles move to an area with higher temperature. In another embodiment, the probe particles move to an area with lower temperature.

The solution can contain other ingredients such as fetal bovine serum (FBS) and/or polyethylene glycol (PEG). In one embodiment, PEG can be added into the solution to increase the difference in the speed of the motion towards the hotter areas between the probe particles and the nucleic acids. The increased difference in the speed of motion increases the difference in the intensity of fluorescence between a contrast area and a predetermined area. The concentration of PEG can be more than 0.1 wt %, preferably, more than 1 wt %, 2 wt %, 3 wt %, 4 wt %, 5 wt %, 6 wt %, 7 wt %, 8 wt %, 9 wt %, and 10 wt %, more preferably, more than 15-20 wt %. In another embodiment, the accumulation of nucleic acids is inhibited, when the concentration of PEG or salt is too high.

Referring to FIG. 1, step S103, a temperature control system is provided to create a temperature gradient in the solution.

The temperature control system of the invention can provide a 2-dimensional (2-D) or 3-dimensional (3-D) temperature gradient by contact or non-contact heating or cooling. The heating element of the invention is not limited. In the following, a non-limiting list of heating elements, which may preferably be used with the invention, will be briefly discussed.

The heating element can be a light-emitting device including a laser, halogen lamp, tungsten lamp, xenon lamp, mercury lamp, and light-emitting diode. These devices can have various constructions (e.g., gas, chemical, and infrared (IR) laser diode) to generate the energy beam. For example, the devices can have a power rating in a range from about 1 W to about 10 W. In one embodiment, the device can be a solid-state laser. The heating element can generate one or more energy beams. The beam parameters may define a wavelength. The heating elements can generate a visible light, near infrared light, infrared light, far infrared light or ultraviolet light.

In another embodiment, the heating element is a typical ohmic heating device. The typical heating element converts a current flow into heat through the process of ohmic heating. Electrical current running through the element encounters resistance, resulting in heating of the element. Bare wires or ribbons, either straight or coiled may be used. Any kind of printed metal/ceramic tracks deposited in/on the heating elements may be used. Further examples for heating elements include heating plates made of ITO (indium tin oxides) or transparent polymers, optically transparent and electrically conductive materials, or microstructures made of electrically conductive but not optically transparent materials such as gold, platinum, and silver. The heating elements may provide either a homogeneous temperature distribution or a spatial temperature gradient in a 2-D or 3-D space. The heating element may be coated with a layer of electrical insulation material, such as polymers and glass, in order to suppress electrochemical reactions. The temperature control system can generate a temperature gradient suitable for the probe particles, labeled molecules, and desired target molecules.

In addition to heating, the temperature gradient can also be achieved by cooling. The range of the temperature gradient is about 10° C. to 60° C., 20° C. to 50° C., 25° C. to 40° C., 10° C. to 25° C., preferably, 25° C. to 35° C.

Referring to FIG. 1, step S105, the expression level of the labeled molecules in a predetermined area and a contrast area is determined.

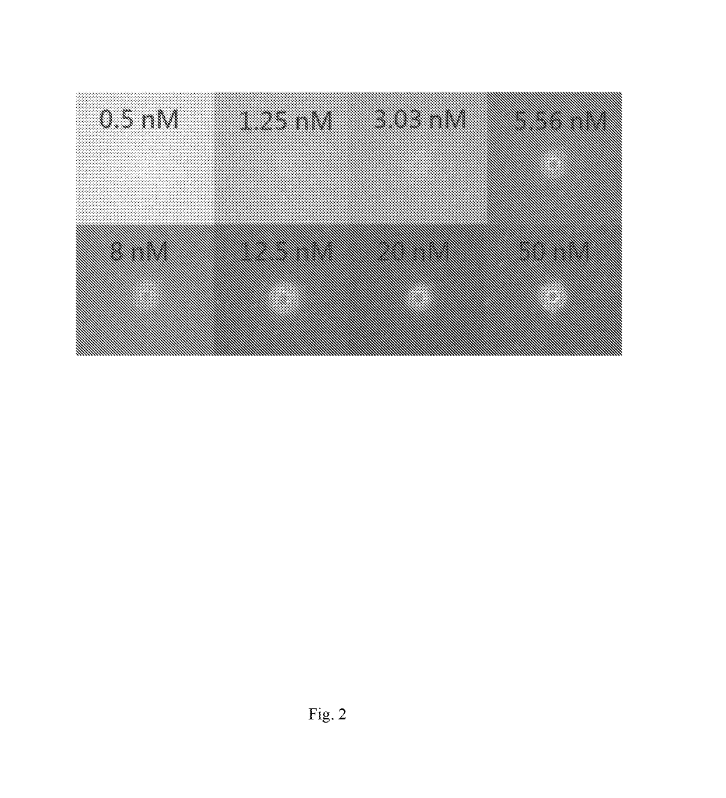

The difference in the expression level of the labeled molecules between the predetermined area and the contrast area changes with the concentration of the target molecules. Referring to FIG. 2, the DNA is detected by a sandwich method, and the concentration of DNA was 0.5 nM, 1.25 nM, 3.03 nM, 5.56 nM, 8 nM, 12.5 nM, 20 nM and 50 nM, respectively. FIG. 2 shows that the accumulation level of the labeled molecules in the predetermined area increases with the concentration of the target molecules. Thus, the concentration of the target molecules can be determined based on the expression level of the labeled molecules.

The “predetermined area” of the invention refers to an area surrounding the heated region (control region). Depending on different needs, samples, and heating methods, an area surrounding the heated region can be defined as a predetermined area of the invention, and the expression level of the labeled molecules is determined in the predetermined area. For example, if the labeled molecule contains fluorescent proteins, the fluorescence intensity is detected in the predetermined area.

At the same time, an area away from the heated region (control region) can be defined as a contrast area of the invention. The average intensity of the free labeled molecules (unbound labeled molecules) in the contrast area is determined as a background value using the same or a similar method.

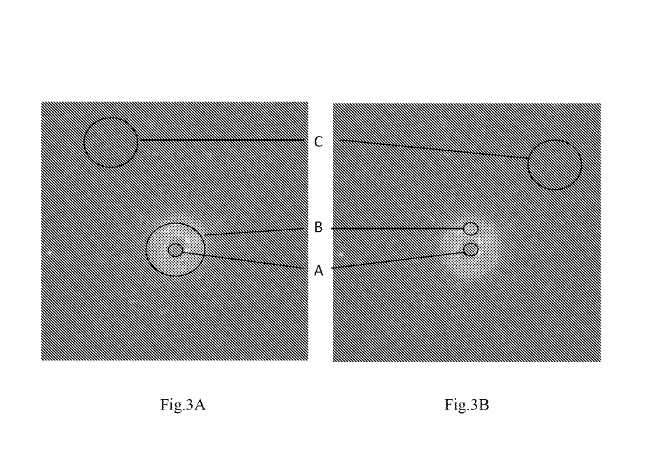

Referring to FIG. 3A-B, a heated region (control region) A is provided, and an area surrounding the heated region (control region) is appropriately selected as the predetermined area B. Additionally, an area away from the heated region (control region) is selected as the contrast area C.

The shape of the predetermined area and the contrast area is not limited and can be circular, rectangular or any shape depending on the heating or analysis methods. In one embodiment, the predetermined area is a circular area. The size of the predetermined area and the contrast area is also not limited. One skilled in the art can select a suitable size depending on the heating or analysis methods.

Referring to FIG. 1, step S107, the difference in the expression level of the labeled molecules between the predetermined area and the contrast area is analyzed to determine the result.

One skilled in the art can use suitable equipment (e.g., optical microscope, fluorescence microscope, and confocal microscope) to observe the fluorophores or dyes linked to labeled molecules. For example, if the labeled molecules are linked to fluorescence proteins, an epifluorescence microscope can be used.

Difference in fluorescence intensity=(fluorescence intensity of predetermined area−fluorescence intensity of contrast area)÷fluorescence intensity of contrast area

According to the standard curve, the concentration of the target molecules can be determined based on the fluorescence intensity.

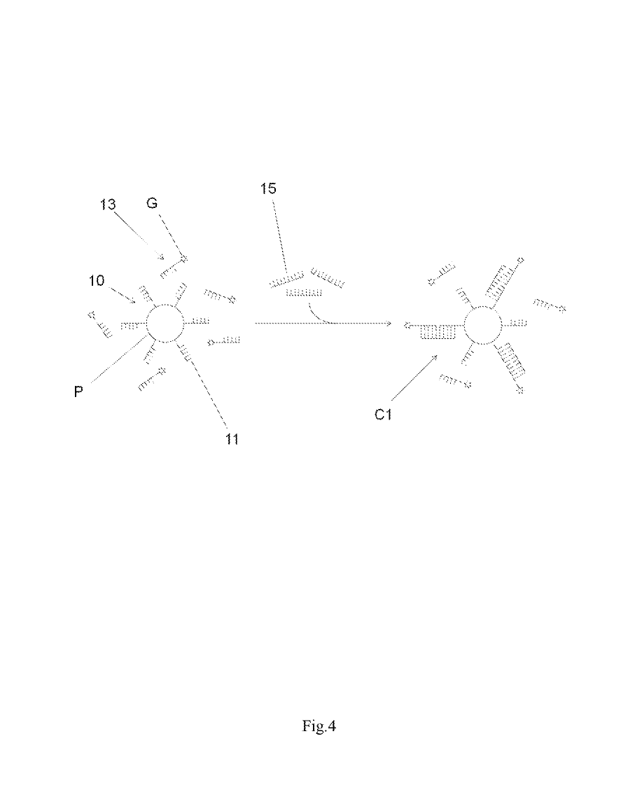

Referring to FIG. 4, in the first aspect of the invention, the probe particle 10 is a particle P linked to the probe 11, and the labeled molecule 13 is linked to the fluorescent molecule G. The probe 11 and the labeled molecule 13 can bind to the target molecule (nucleic acids) 15 to form the complex C1. After heating, the complex C1 moves towards a heated region. If the labeled molecule 13 is not linked to the probe 11, the migration of the labeled molecule 13 is slow. Conversely, if the labeled molecule 13 is linked to the probe 11, the migration of the labeled molecule 13 is rapid. Therefore, the accumulation level of the fluorescent molecule G (labeled molecule 13) increases with the increased concentration of the target molecule 15.

In the present invention, the probe particle 10 is used to increase the difference in thermophoresis between the molecular complex and the free labeled molecule. The method of the invention can improve the accuracy of the quantification of the target molecules using thermophoresis.

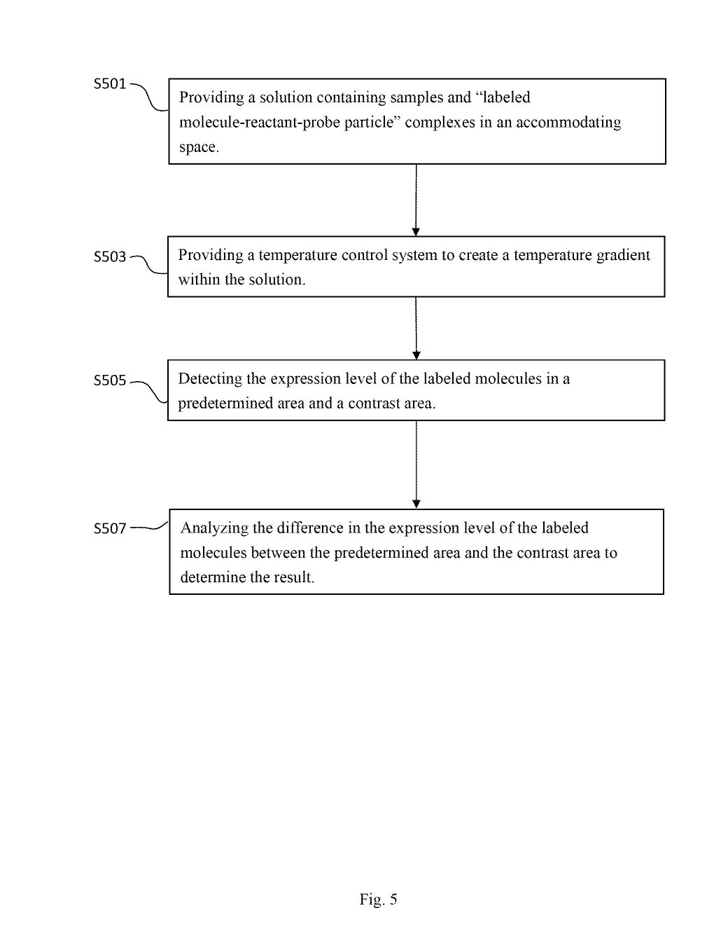

In another aspect of the invention, the invention also provides a method for analyzing a target molecule in a sample, as shown in FIG. 5.

Referring to FIG. 5, step S501, a solution is provided in an accommodating space, wherein the solution contains samples and “labeled molecule-reactant-probe particle” complexes.

The “reactant” of the invention refers to, but is not limited to, a DNA molecule, a RNA molecule, or a protein. It should be noted that the reactant of the invention can bind to a labeled molecule and a probe particle to form a complex.

Referring to FIG. 5, step S503, a temperature control system is provided to create a temperature gradient in the solution.

Referring to FIG. 5, step S505, the intensity level of the labeled molecule in a predetermined area and a contrast area is determined

Referring to FIG. 5, step S507, the difference in the expression level of the labeled molecules between the predetermined area and the contrast area is analyzed to determine the result.

In the aspect of the invention (FIG. 5), the speed or direction of the motion of the molecular complexes in a temperature gradient is different from that of the free labeled molecules, and the affinity of the reactant to the target molecule is higher than that of the labeled molecule and the probe particle. Accordingly, the aspect of the invention is distinct from the first aspect of the invention (FIGS. 1 and 4). The reactant can bind to the target molecule to disrupt the complex in the presence of the target molecule.

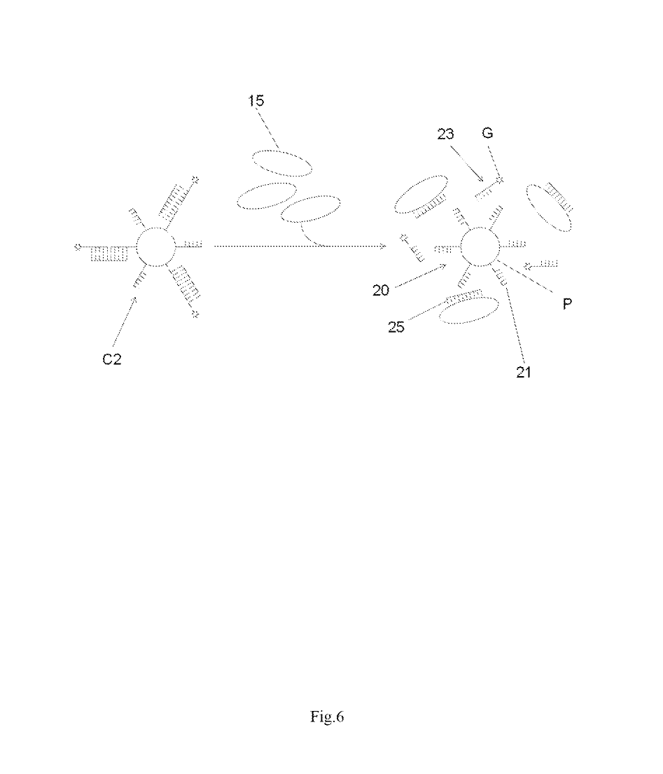

Referring to FIG. 6, the reactant 25 can bind to the labeled molecule 23 and the probe particle 21 to form the complex C2, wherein the probe 21 is linked to the particle P, and the labeled molecule 23 is linked to the fluorescent molecule G. When the target molecule 15 is present in the solution, the reactant 25 can bind to the target molecule 15 to disrupt the complex C2 because the affinity of target molecule 15 to the reactant 25 is higher than that to the labeled molecule 23 and the probe 21. When the amount of the target molecule 15 is high, the accumulation of the free labeled molecules at the heated region by thermophoresis is not apparent because the thermophoretic motion of the free labeled molecule 23 is slow. If the amount of the target molecules 15 is low, many of the labeled molecules 23 can bind to the probe 21. Because the probe particle 20 move towards the heated region fast, the accumulation of the fluorescent molecule G that is indirectly linked to the particle P is high. Therefore, the concentration of target molecule 15 can be determined according to the accumulation level of the fluorescence molecule G.

As mentioned above, the method of the invention can be used to detect nucleic acids and proteins. The probe particle 20 of the present invention can improve the accuracy of the quantification of the target molecules using thermophoresis.

### EXAMPLE

### Example 1

### Preparation of Samples for the Detection of DNA (Sandwich Method)

20 nm of gold nanoparticles were mixed with thiol-modified DNA (DNA_{—}1) in the ratio 1:1,000 in 10 mM of phosphate buffer containing 0.5 M NaCl. The concentration of the gold nanoparticles was 1.2 nM. DNA_{—}1 molecules were bound to the surface of the gold nanoparticles through thiol group. The unbound DNA_{—}1 molecules were removed by certification, and the gold nanoparticle solution was concentrated to reach a concentration of 2.3 nM.

50 μl of the resulting solution was mixed with 1 μl of FITC-modified DNA (DNA_{—}2, 1 μM) in room temperature. After mixing, the concentration of DNA_{—}2 was 19.6 nM.

Several known concentrations of DNA (DNA_{—}3) solutions were prepared to obtain a calibration curve for DNA quantification. The DNA_{—}3 was diluted with fetal bovine serum (FBS) to reach final concentrations ranging from 0.5 nM to 50 nM (1 μl of the stock solution were diluted 20, 50, 80, 125, 180, 330, 800, and 2000 times). A part of the sequence of DNA_{—}3 was complementary to the sequence of DNA_{—}1, which was linked to the gold nanoparticles. The other part of the sequence of DNA_{—}3 was complementary to the sequence of DNA_{—}2. When DNA_{—}3 was present in the solution, DNA_{—}1, DNA_{—}2, and DNA_{—}3 would bind together through base pairing. The sequences of the DNA are shown in Table 1.

TABLE 1

| SEQ ID NO | No. | Sequence |
|---|---|---|
| SEQ ID NO: 1 | DNA_1 | Thiol-AAAAAAAACACAACACCCAA |
| SEQ ID NO: 2 | DNA_2 | CACAACCAACCCCAAAAAAA-FITC |
| SEQ ID NO: 3 | DNA_3 | TGGGGTTGGTTGTGTTGGGTGTTGTGTTT |

6 μl of the mixture that contained DNA_{—}1 and DNA_{—}2 was mixed with 1 μl of DNA_{—}3 to have a solution containing DNA_{—}1, DNA_{—}2, and DNA_{—}3. After mixing, the concentration of gold nanoparticles was changed to 2.0 nM, and the concentration of DNA_{—}2 was changed to 16.8 nM. After mixing the three kinds of DNA, some fluorescent DNA (DNA_{—}2) molecules were linked to the surfaces of the gold nanoparticles in the presence of DNA_{—}3, and the amount of the DNA_{—}2 linked to the gold nanoparticles increased with the increased amount of DNA 3.

3 μl of 50 wt % polyethylene glycol (PEG, 10,000 MW) was added to the solution. After mixing, the mass fraction of PEG was 15 wt %. The concentration of DNA_{—}2 was 11.8 nM, and the concentration of the gold nanoparticles was 1.4 nM. The concentration of FBS was 10%. The mixture was mixed for 10 minutes and was then analyzed using thermophoresis.

### Example 2

### Detection of Molecules Using Thermophoresis

A cover slip coated with a thin chromium layer (40 nm) was prepared, and two pieces of double-sided tape was attached to the surface of the chromium layer. The distance between the two pieces of double-sided tape was about 2 mm. An uncoated cover slip was put on the top of the chromium layer and the double-sided tape to form a microchamber between the two cover slips. The microchamber could accommodate an aqueous sample that has a volume of about 3 μl. The sample was injected into the microchamber for analysis.

A temperature gradient was provided by near infrared laser light (Nd:YAG, 1064 nm, power was 8 mW before an objective lens) focused on the chromium layer using a 20× objective lens with N.A. 0.45. The temperature of the solution at the focal point was increased to 31° C. After laser exposure, the fluorescent DNA (DNA_{—}2) was not uniformly distributed around the heated region because of thermophoresis. Fluorescence was observed using a charge coupled device (CCD) and an epifluorescence microscope (Olympus, BX51M) with a 20× objective lens (N.A. 0.75). The exposure time of the camera was 0.5 seconds. The laser light source was turned on for 5 minutes and then fluorescence images were acquired for detection.

When fluorescence images were analyzed, a circular area centered at the focal point with a diameter of 20 pixels was selected. The average fluorescence intensity of the circular area was determined as I1. In addition, a rectangular area with a length of 170 pixels and a width of 90 pixels at a distance of 20 pixels from the focal point was selected. The average fluorescence intensity of the rectangular area was determined as I2. The relative change in fluorescence intensity around the focal point was calculated using a formula as follows:

Relative change in fluorescence intensity=(I1−I2)/I2×100% (Formula I)

The relative change in fluorescence intensity was determined with samples that contained DNA_{—}3 of various concentrations to obtain a calibration curve for the quantification of DNA. Referring to FIG. 6, the accumulation level of the fluorescent molecules increased with the DNA_{—}3 concentration.

### Example 3

### Preparation of Samples for the Detection of Proteins (Competition Method)

40-nm gold nanoparticles were mixed with thiol-modified DNA (DNA_{—}1) in the ratio 1:400 in 1× phosphate buffered saline (PBS). After mixing, the concentration of the gold nanoparticles was 0.3 nM, and the concentration of DNA_{—}1 was 119.2 nM. DNA_{—}1 molecules were bound to the surface of the gold nanoparticles through thiol group.

The unbound DNA_{—}1 molecules were removed by certification, and the gold nanoparticle solution was concentrated to reach a concentration of 0.6 nM.

Interferon-γ (IFN-γ) aptamers were used as the probes (DNA_{—}3) for the detection of IFN-γ. After mixing 1 μl of 10 μM DNA_{—}3, 1 μl of 10 μM FITC modified DNA (DNA_{—}2), and 8 μl of 1×PBS, the concentration of DNA_{—}2 and DNA_{—}3 was 1 μM.

Several known concentrations of IFN-γ solutions were prepared to obtain a calibration curve for IFN-γ quantification. The IFN-γ was diluted with 1×PBS to reach final concentrations ranging from 0.6 nM to 300 nM.

6 μl of the mixture that contained DNA_{—}1, DNA_{—}2, and DNA_{—}3 was mixed with 1 of IFN-γ to have a solution containing DNA_{—}1, DNA_{—}2, DNA_{—}3, and IFN-γ.

3 μl of 50 wt % PEG (10,000 MW) was added to the solution. After mixture, the mass fraction of PEG was 15 wt %. The concentration of DNA_{—}2 and DNA_{—}3 was 11.8 nM, and the concentration of the gold nanoparticles was 0.4 nM. The mixture was mixed for 10 minutes and was then analyzed using thermophoresis.

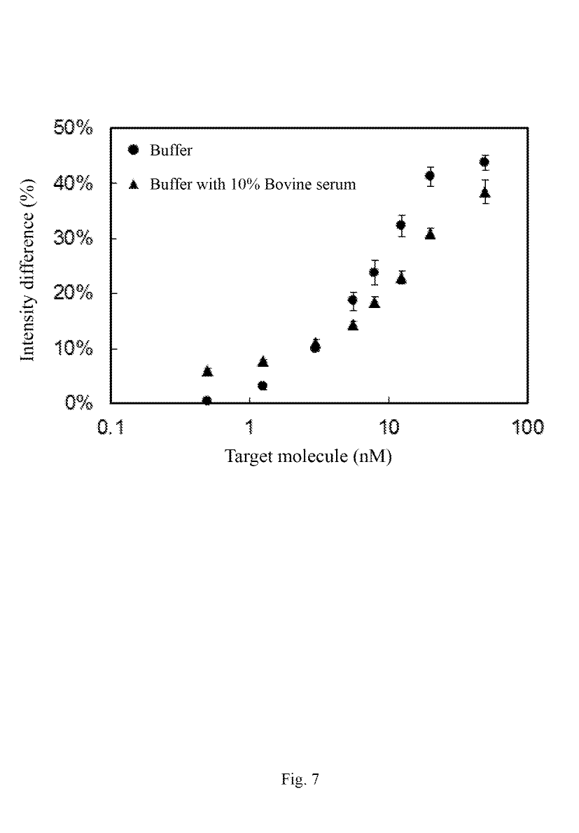

The processes of Example 2 and Formula I were used to calculate the relative change in fluorescence intensity. Referring to FIG. 7, when DNA_{—}3 bound to IFN-γ, DNA_{—}2 dissociated from the gold nanoparticles. Therefore, the amount of DNA_{—}2 linked to the gold nanoparticles decreased with the increased amount of IFN-γ.

While the invention has been described by examples and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation to encompass all such modifications and similar arrangements.

What is claimed is:

1. A method for analyzing a target molecule in a sample by using thermophoresis, comprising:
providing a solution comprising samples, labeled molecules, and probe particles in an accommodating space;
providing a temperature control system in a control region of the accommodating space to create a temperature gradient within the solution;
detecting an expression level of the labeled molecules in a predetermined area and a contrast area; and
analyzing the difference in the expression level of the labeled molecules between the predetermined area and the contrast area to determine a result,
wherein the probes and the labeled molecules are linked to the target molecules to form “probe particle-target molecule-labeled molecule” complexes if the sample contains the target molecules, and the direction or speed of the motion of the molecular complexes in the temperature gradient is different from that of free labeled molecules.

2. The method according to claim 1, wherein the probe particle is a nanoparticle with at least one probe attached on a surface thereof.

3. The method according to claim 2, wherein the probe is a DNA molecule, a RNA molecule or an antibody.

4. The method according to claim 2, wherein the nanoparticle is sensitive to thermophoresis.

5. The method according to claim 2, wherein the nanoparticle is a metal, plastic, glass, oxide or semiconductor nanoparticle.

6. The method according to claim 5, wherein the nanoparticle is a gold nanoparticle.

7. The method according to claim 1, wherein the target molecule is a DNA molecule, a RNA molecule, a protein, an organic or inorganic small molecule.

8. The method according to claim 1, wherein the labeled molecule is a DNA molecule, a RNA molecule, or an antibody labeled with fluorophores or dyes.

9. The method according to claim 8, wherein the expression level of the labeled molecules is an intensity of fluorescence.

10. The method according to claim 1, wherein the heating or cooling is conducted by a temperature control system.

11. The method according to claim 10, wherein the heating is conducted by an electrode or a light emitting device of the temperature control system.

12. The method according to claim 1, wherein the accommodating space is a microchamber or a capillary tube.

13. A method for analyzing a target molecule using thermophoresis, comprising:
providing a solution comprising a samples and “labeled molecule-reactant-probe particle” complexes in an accommodating space;
providing a temperature control system in a control region of the accommodating space to create a temperature gradient within the solution;
detecting an expression level of the labeled molecules in a predetermined area and a contrast area; and
analyzing the difference in the expression level of the labeled molecules between the predetermined area and the contrast area to determine a result,
wherein the direction or speed of the motion of the molecular complexes in the temperature gradient is different from that of the free labeled molecules, and reactants bind to the target molecules present in the solution and disrupt the complexes because a binding affinity of the reactants to the target molecules is higher than that to the labeled molecules and the probe particles.

14. The method according to claim 13, wherein the probe particle is a nanoparticle with at least one probe attached on a surface thereof.

15. The method according to claim 14, wherein the probe is a DNA molecule, a RNA molecule, or an antibody.

16. The method according to claim 14, wherein the nanoparticle is sensitive to thermophoresis.

17. The method according to claim 14, wherein the nanoparticle is a metal, plastic, glass, oxide or semiconductor nanoparticle.

18. The method according to claim 17, wherein the nanoparticle is a gold nanoparticle.

19. The method according to claim 13, wherein the target molecule is a DNA molecule, a RNA molecule, or a protein.

20. The method according to claim 13, wherein the labeled molecule is a DNA molecule, a RNA molecule, or an antibody labeled with fluorophores or dyes.

21. The method according to claim 20, wherein the expression level of the labeled molecule is the intensity of fluorescence.

22. The method according to claim 13, wherein the heating or cooling is conducted by a temperature control system.

23. The method according to claim 22, wherein the heating is conducted by an electrode or a light emitting device of the temperature control system.

24. The method according to claim 13, wherein the accommodating space is a microchamber or a capillary tube.


SEQUENCE LISTING (3 sequences)

SEQ ID NO: 1 - DNA, 20 bp, Artificial
  Syntheized Sequence
aaaaaaaacacaacacccaa

SEQ ID NO: 2 - DNA, 20 bp, Artificial
  Synthesized Sequence
cacaaccaaccccaaaaaaa

SEQ ID NO: 3 - DNA, 29 bp, Artificial
  Synthesized Sequence
tggggttggttgtgttgggtgttgtgttt